(19) United States
(12) Patent Application Publication
(10) Pub. No.: US 2019/0270980 A1
(43) Pub. Date: Sep. 5, 2019

(54) TREATING CANCER

(71) Applicant: Mayo Foundation for Medical Education and Research, Rochester, MN (US)

(72) Inventors: Yasuhiro Ikeda, Rochester, MN (US); Yuichi Machida, Rochester, MN (US); Jason M. Tonne, Rochester, MN (US); Salma G. Morsy, Rochester, MN (US)

(73) Assignee: Mayo Foundation for Medical Education and Research, Rochester, MN (US)

(21) Appl. No.: 16/320,186

(22) PCT Filed: Jul. 24, 2017
(86) PCT No.: PCT/US2017/043452

§ 371 Date: Jan. 24, 2019

Related U.S. Application Data
(60) Provisional application No. 62/366,341, filed on Jul. 25, 2016.

(51) Int. Cl. C12N 15/10; C12N 15/86; A61P 35/00; A61K 9/00

(52) U.S. Cl. CPC C12N 15/102; C12N 15/86; C12N 2750/14143; A61K 9/0019; A61P 35/00

(57) ABSTRACT

This document relates to methods and materials for treating cancer. For example, methods and materials for using CRISPR/Cas9 systems to treat cancer are provided.

### CROSS-REFERENCE TO RELATED APPLICATIONS

This application claims the benefit of U.S. Provisional Application Ser. No. 62/366,341, filed Jul. 25, 2016. The disclosure of the prior application is considered part of (and is incorporated by reference in) the disclosure of this application.

### BACKGROUND

### 1. Technical Field

This document relates to methods and materials for treating cancer. For example, this document provides methods and materials for using Clustered Regularly Interspaced Short Palindromic Repeats (CRISPR)-Cas9 systems to treat cancer.

### 2. Background Information

Cancer is the second-leading cause of death in the United States. One example of cancer is breast cancer, which develops from breast tissue and is the most common invasive cancer in women. Breast cancer is usually treated with surgery, which may be followed by chemotherapy or radiation therapy, or both chemotherapy and radiation therapy. However, there is no effective therapy for certain types of cancers, such as pancreatic cancer.

### SUMMARY

This document provides methods and materials for treating cancer. For example, this document provides methods and materials for using CRISPR/Cas9 systems to treat cancer. As described herein, gene editing techniques such as those involving the use of a CRISPR/Cas9 system can be designed to cleave a cell cycle gene (e.g., a CDK1 nucleic acid and/or a PCNA nucleic acid) and/or a repetitive nucleic acid sequence (e.g., an Alu nucleic acid sequence, an HERV-K nucleic acid sequence, and/or an HERV-9 nucleic acid sequence). Cleavage of a cell cycle gene and/or a repetitive nucleic acid sequence within cancer cells can reduce cancer cell proliferation and/or induce cancer cell death. In some cases, a single viral vector such as an adeno-associated virus (AAV) vector can be used to deliver both nucleic acid encoding the Cas9 component and the targeting guide RNA of a CRISPR/Cas9 system. In some cases, the Cas9 component can be a Staphylococcus aureus Cas9 (saCas9).

In general, one aspect of this document features a nucleic acid construct including a nucleic acid encoding a Cas9 polypeptide and a nucleic acid encoding a targeting guide RNA, where the targeting guide RNA targets a cell cycle gene or a repetitive nucleic acid sequence. The Cas9 polypeptide can be a saCas9 polypeptide. The targeting guide RNA can target the cell cycle gene. The cell cycle gene can be CDK1 or PCNA1. The targeting guide RNA can target the repetitive nucleic acid sequence. The repetitive nucleic acid sequence can be an Alu nucleic acid sequence, an HERV-K nucleic acid sequence, or an HERV-9 nucleic acid sequence.

In another aspect, this document features a viral vector including a nucleic acid encoding a Cas9 polypeptide and a nucleic acid encoding a targeting guide RNA, where the targeting guide RNA targets a cell cycle gene or a repetitive nucleic acid sequence. The Cas9 polypeptide can be a saCas9 polypeptide. The targeting guide RNA can target the cell cycle gene. The cell cycle gene can be CDK1 or PCNA1. The targeting guide RNA can target the repetitive nucleic acid sequence. The repetitive nucleic acid sequence can be an Alu nucleic acid sequence, an HERV-K nucleic acid sequence, or an HERV-9 nucleic acid sequence. The viral vector can be an AAV.

In another aspect, this document features a method for reducing the number of cancer cells within a mammal having cancer. The method includes, or consists essentially of, administering to a mammal a nucleic acid construct including a nucleic acid encoding a Cas9 polypeptide and a nucleic acid encoding a targeting guide RNA, where the targeting guide RNA targets a cell cycle gene or a repetitive nucleic acid sequence, or administering to a mammal a viral vector including a nucleic acid encoding a Cas9 polypeptide and a nucleic acid encoding a targeting guide RNA, where the targeting guide RNA targets a cell cycle gene or a repetitive nucleic acid sequence.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 contains gRNA-recognition region sequences showing site-directed gene deletion sites in (a) CDK1 vector-treated cells, and (b) PCNA vector-treated cells. Sequences shown in FIG. 1A include the CDK1 target region (underlined; SEQ ID NO:25), the original CDK1 sequence (SEQ ID NO:26), and sequences with deletions observed in individual clones (SEQ ID NOs: 27-36 from top to bottom). Sequences shown in FIG. 1B include the PCNA target region (underlined; SEQ ID NO:37), the original PCNA sequence (SEQ ID NO:38), sequences with deletions observed in individual clones (SEQ ID NOs: 39-48 from top to bottom).

FIG. 2 is a graph plotting cell proliferation of untreated HeLa cells or HeLa cells treated with a CRISPR/saCas9 system designed to cleave CDK1 nucleic acid (Cas9-CDK1) or HeLa cells treated with a CRISPR/saCas9 system designed to cleave PCNA nucleic acid (Cas9-PCNA).

FIG. 3 is a graph plotting cell proliferation of untreated HT1080 cells or HT1080 cells treated with a CRISPR/saCas9 system designed to cleave CDK1 nucleic acid (Cas9-CDK1) or HT1080 cells treated with a CRISPR/saCas9 system designed to cleave PCNA nucleic acid (Cas9-PCNA).

FIG. 4 contains photographs of (a) untreated HeLa cells or HT1080 cells (left panels), (b) HeLa cells or HT1080 cells after being treated with a CRISPR/saCas9 system designed to cleave PCNA nucleic acid (Cas9-PCNA) for four consecutive days (center panels), and (c) HeLa cells or HT1080 cells after being treated with a CRISPR/saCas9 system designed to cleave CDK1 nucleic acid (Cas9-CDK1) for four consecutive days (right panels).

FIG. 5 contains photographs of U251 cells after treatment with an AAV-CRISPR/saCas9 (AAV-saCRISPR) system designed to cleave a GFP control, CDK1, HERV9-1, HERV9-2, or HERVK-2.

FIG. 6 is a listing of the nucleic acid sequence of pX601-AAV-CMV::NLS-SaCas9-NLS-3xHA-bGHpA;U6::BsaI-sgRNA plasmid.

FIG. 7 is a graph plotting in vivo tumor volume of mice treated with control vectors (AAV2-MCS) or treated with vectors expressing Cas9 and CDK1 (AAV2-Cas9-CDK1).

FIG. 8 is a survival curve of mice treated with control vectors (AAV2-MCS) or treated with vectors expressing Cas9 and CDK1 (AAV2-Cas9-CDK1).

FIG. 9 is a graph plotting in vivo tumor volume of mice treated with control vectors (AAV2-MCS) or treated with vectors expressing Cas9 and CDK1 (AAV2-Cas9-CDK1), vectors expressing Cas9 and HERV9 (AAV2-Cas9-HERV9), or vectors expressing Cas9 and HERVK (AAV2-Cas9-HERVK).

### DETAILED DESCRIPTION

This document provides methods and materials for treating cancer. For example, this document provides methods and materials for using CRISPR/Cas9 systems designed to cleave a cell cycle gene (e.g., a CDK1 nucleic acid and/or a PCNA nucleic acid) and/or a repetitive nucleic acid sequence (e.g., an Alu nucleic acid sequence, an HERV-K nucleic acid sequence, and/or an HERV-9 nucleic acid sequence) to treat cancer, to reduce the proliferation of cancer cells, and/or to reduce the number of cancer cells within a mammal. As described herein, cleavage of a cell cycle gene within cancer cells can result in reduced cancer cell proliferation and/or induced cancer cell death via, for example, the interference with cell division. Cleavage of a repetitive nucleic acid sequence within cancer cells can result in reduced cancer cell proliferation and/or induced cancer cell death via, for example, genome fragmentation.

Any appropriate mammal can be treated as described herein. Examples of mammals that can be administered a CRISPR/Cas9 system designed to cleave a cell cycle gene and/or a repetitive nucleic acid sequence include, without limitation, humans, non-human primates, monkeys, bovine species, pigs, horses, dogs, cats, sheep, goat, and rodents.

Any appropriate cancer can be treated using the methods and materials described herein. For example, breast, lung, brain, pancreatic, prostate, liver, and skin cancer, or hematopoietic malignancy, such as leukemia and myeloma, can be treated by administering a CRISPR/Cas9 system designed to cleave a cell cycle gene (e.g., a CDK1 nucleic acid and/or a PCNA nucleic acid) and/or a repetitive nucleic acid sequence (e.g., an Alu nucleic acid sequence, an HERV-K nucleic acid sequence, and/or an HERV-9 nucleic acid sequence). In some cases, the number of breast, lung, brain, pancreatic, prostate, liver, and skin cancer cells present within a mammal (e.g., a human) can be reduced by administering a CRISPR/Cas9 system designed to cleave a cell cycle gene (e.g., a CDK1 nucleic acid and/or a PCNA nucleic acid) and/or a repetitive nucleic acid sequence (e.g., an Alu nucleic acid sequence, an HERV-K nucleic acid sequence, and/or an HERV-9 nucleic acid sequence).

The Cas9 component of a CRISPR/Cas9 system designed to cleave a cell cycle gene and/or a repetitive nucleic acid sequence can be any appropriate Cas9 such as those described elsewhere (Cong et al., 2013 Science, 339:819-823). In some cases, the Cas9 of a CRISPR/Cas9 system designed to cleave a cell cycle gene and/or a repetitive nucleic acid sequence can be a Staphylococcus aureus Cas9 (saCas9). The nucleic acid or polypeptide sequence of an saCas9 is described elsewhere (Ran et al., Nature, 520:186-191 (2015)).

In some cases, the Cas9 of a CRISPR/Cas9 system designed to cleave a cell cycle gene and/or a repetitive nucleic acid sequence can be replaced with another functional domain capable of carrying out gene editing. For example, Zinc finger nucleases (ZFNs) or TALE nucleases (TALENs), can be used in place of Cas9 to design gene editing systems with targeting guide RNA to cleave a cell cycle gene and/or a repetitive nucleic acid sequence. The nucleic acid or polypeptide sequence of such genome editing molecules can be as described elsewhere (Mani et al., Biochemical and Biophysical Research Communications, 335:447-457, 2005; Campbell et al., Circulation Research, 113:571-587, 2013).

A CRISPR/Cas9 system provided herein can be designed to target any appropriate cell cycle gene. Examples of cell cycle genes that can be targeted as described herein include, without limitation, CDK1 nucleic acids, PCNA nucleic acids, CDK2 nucleic acids, CCNB1 nucleic acids, CCNE1 nucleic acids, and ORC1 nucleic acids. In some cases, a cell cycle gene that is targeted using a CRISPR/Cas9 system described herein can be a human CDK1 nucleic acid, a human PCNA nucleic acid, a human CDK2 nucleic acid, a human CCNB1 nucleic acid, a human CCNE1 nucleic acid, and a human ORC1 nucleic acid. One example of a human CDK1 nucleic acid is set forth in GenBank Accession No. NP_001777.1 (GI No. 4502709). One example of a human PCNA nucleic acid is set forth in GenBank Accession No. NP_002583.1 (GI No. 4505641).

One example of a human CDK2 nucleic acid is set forth in GenBank Accession No. NP_001789.2 (GI No. 16936528).

One example of a human CCNB1 nucleic acid is set forth in GenBank Accession No. NP_114172.1 (GI No. 14327896).

One example of a human CCNE1 nucleic acid is set forth in GenBank Accession No. NP_001229.1 (GI No. 17318559).

One example of a human ORC1 nucleic acid is set forth in GenBank Accession No. NP_001177747.1 (GI No. 299890793).

A CRISPR/Cas9 system provided herein can be designed to target any appropriate repetitive nucleic acid sequence, such as retrotransposons. As used herein, “repetitive nucleic acid sequence” refers to a nucleic acid sequence that is at least 22 bases long and endogenously occurs throughout the genome of a cell at least 10 times.

Examples of repetitive nucleic acid sequences that can be targeted as described herein include, without limitation, Alu nucleic acid sequences, HERV-K nucleic acid sequences, HERV-9 nucleic acid sequences, HERV-H nucleic acid sequences, HERV-E nucleic acid sequences, HERV-S nucleic acid sequences, and HERV-HML5 nucleic acid sequences. In some cases, a repetitive nucleic acid sequence that is targeted using a CRISPR/Cas9 system described herein can be a human Alu nucleic acid sequence, a human HERV-K nucleic acid sequence, a human HERV-9 nucleic acid sequence, a human HERV-H nucleic acid sequence, a human HERV-E nucleic acid sequence, a human HERV-S nucleic acid sequence, and a human HERV-HML5 nucleic acid sequence. Examples of Alu nucleic acid sequences that can be targeted using a CRISPR/Cas9 system provided herein include, without limitation, GCCAGATGCGGTGGCTCACGCCTGTAATCCCAGCACTTTGG-GAGGCTGAGGCAGTTGGATCACCTGAGGTCAGGAATTAGCACCACTGCAC TCCAGCCTAGGCGACGAGAGCAAAACTCTGTCTCAAAAAAAAAAAAAAA GAAAGAAAGAAAAAAGAAAGGGCTAGGAGCTACAA (SEQ ID NO:1), CCT-GTAATCCCAGCACTTTGGGAGGC (SEQ ID NO:2), AAAAGTAAAAAGA-GGGGCCAGGCACAGTGGCTCAGCCTGTAATCCCAGCACTTTGGGAGGCTG AGGTGGGCAGGATCACCTGAGCTCGGGAAGTTGAGGCTAATAGTGGGCTG AGATTGTGCCACTGCACTCCAGCCTGGGTGACAGGGAAGGAGACCCTGTC TCAAA (SEQ ID NO:3), GGCCAGGCATGGTGCTCATCGCCTGTAATC-CCAGCACTTTGGGAGGCCGAGAAAGATGGATGAAGTCAGGAGTTCAAGA CCAGCCTGGGCAACATGGCAGAACCCCGTCTCTACTAAAAATACAAAAAA TTAGCCGGGCGTGGTGGTGGGCGCCTGTAATCCCAGC (SEQ ID NO:4), and GGCCGGGCGCGGTGGCTCACGCCTGTAATCCCAGCACTTTGGGAGGCCGA GGCGGGCGGATCACGAGGTCAGGAGATCGAGACCATCCCGGCTAAAACG GTGAAACCCCGTCTCTACTAAAAATACAAAAATTAGCCGGGCGTAGTGGC GGGCGCCTGTAGTCCCAGCTACTTGGGAGGCTGAGGCAGGAGAATGGCGT GAACCCGGGAGGCGGAGCTTGCAGTGAGCCGAGATCCCGCCACTGCACTC CAGCCTGGGCGACAGAGCGAGACTCCGTCTCAAAAAAAA (SEQ ID NO:5). Examples of HERV-K nucleic acid sequences that can be targeted using a CRISPR/Cas9 system provided herein include, without limitation, CTGTTAATC-TATGACCTTACCCCCAACCCCGTGCTCTCTGAAACG (SEQ ID NO:6) and CCTTAAGAGTCATCACCACTCCCTAATCTCAAGTACCCAGGGACACA (SEQ ID NO:7). Other examples of HERV-K nucleic acid sequences that can be targeted using a CRISPR/Cas9 system provided herein include those set forth in GenBank Accession Nos. Y18890.1 (GI: 5931703), Y17832.2 (GI: 4581240), Y17834.1 (GI: 4185945), or Y17833.1 (GI: 4185941). Examples of HERV-9 nucleic acid sequences that can be targeted using a CRISPR/Cas9 system provided herein include, without limitation, CAGGATGTG-GGTGGGGCCAGATAAGAGAATAAAAGCAGGC (SEQ ID NO:8) and TGCC-CGAGCCAGCAGTGGCAACCCGCTCGGGTCCCCTTCC (SEQ ID NO:9). Other examples of human HERV-K nucleic acid sequences that can be targeted using a CRISPR/Cas9 system provided herein include those set forth in GenBank Accession Nos. AF072495.1 (GI:4262279), AY189679.1 (GI: 30013649), AF064191.1 (GI:

4249626), and EF543088.1 (GI: 151428371).

Any appropriate method can be used to deliver a CRISPR/Cas9 system described herein or nucleic acid encoding a CRISPR/Cas9 system described herein to cancer cells. For example, a CRISPR/Cas9 system described herein can be directly injected into a tumor. In some cases, a viral vector (e.g., an oncolytic viral vector) can be used to deliver nucleic acid encoding a CRISPR/Cas9 system described herein to cancer cells within a mammal (e.g. a human). Examples of viral vectors that can be used to deliver nucleic acid encoding a CRISPR/Cas9 system described herein to cancer cells within a mammal include, without limitation, AAV vectors (e.g. AAV1, AAV2, AAV5, AAV6, AAV8, AAV9, AAV-rh10, vectors with engineered AAV capsid), Adenoviral vectors (e.g. Ad5, Ad6, Ad26), lentiviral vectors (e.g. HIV, FIV, SIV, EIAV), retroviral vectors (e.g. MLV, Foamy viruses), Herpesviral vectors (e.g. HSV, EB, VZV), Pox viral vectors (e.g. vaccinia), baculoviral vectors, vesicular stomatitis viral vectors, Sendai viral vectors, alphaviral vectors, measles viral vectors and Borna disease viral vectors. In some cases, a single AAV vector can be designed to deliver both nucleic acid encoding the Cas9 component (e.g., an saCas9) and the targeting guide RNA of a CRISPR/Cas9 system.

The invention will be further described in the following examples, which do not limit the scope of the invention described in the claims.

### EXAMPLES

### Example 1

### Designing CRISPR/Cas9 Systems that Target CDK1 and PCNA Nucleic Acid

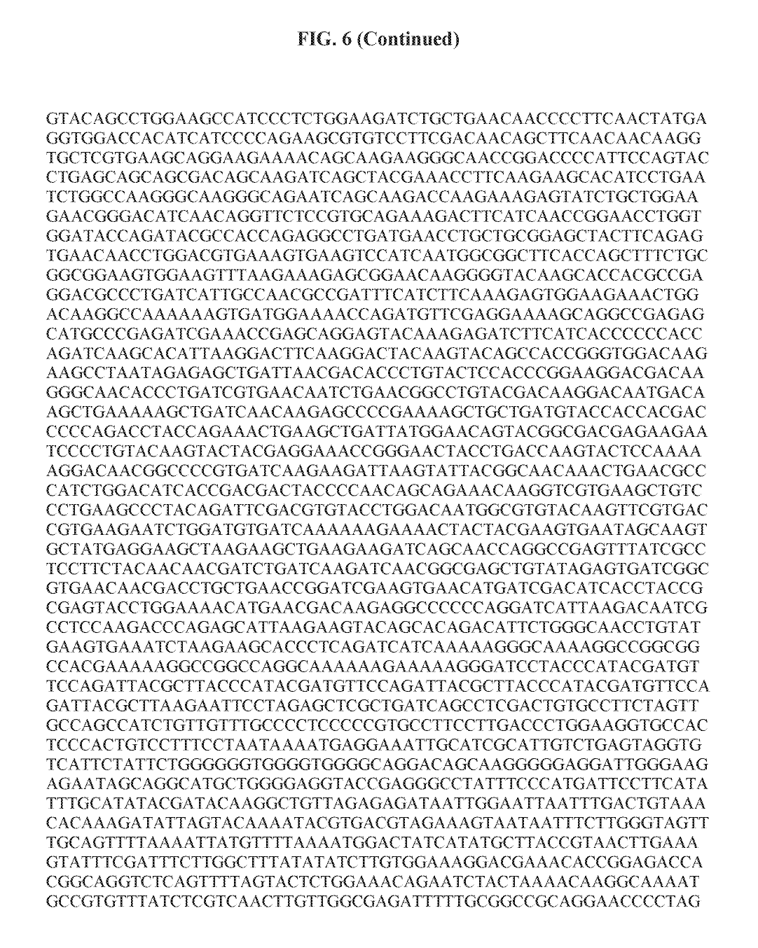

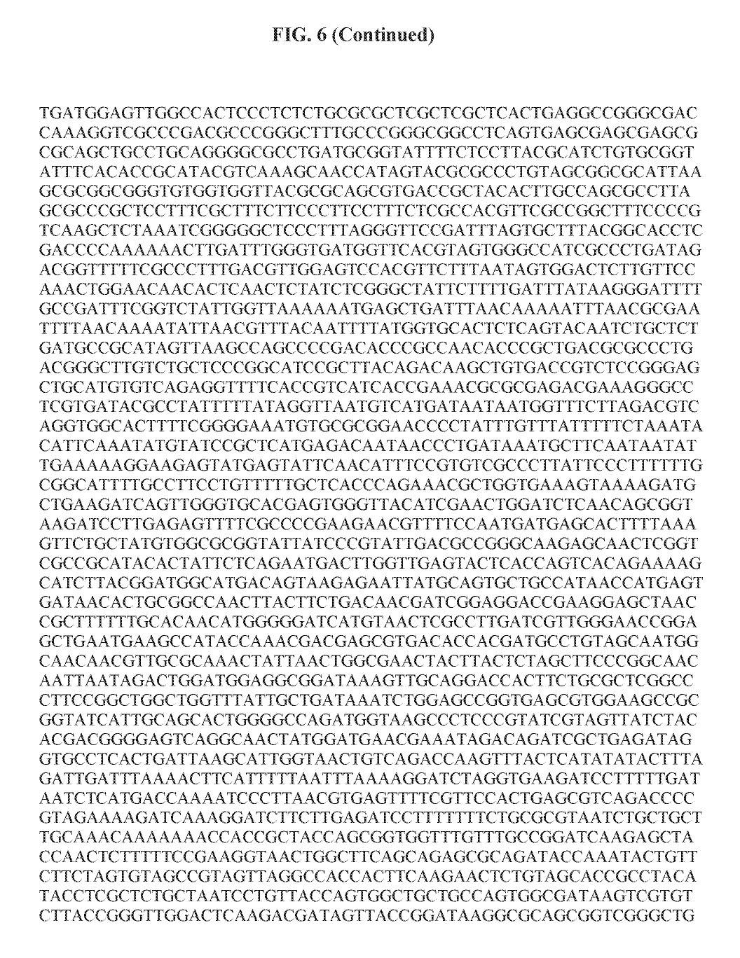

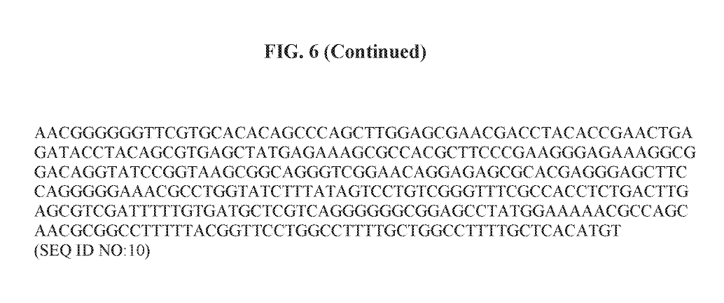

AAV vectors were designed to express an saCas9 polypeptide and a targeting guide RNA of a CRISPR/Cas9 system. The detailed protocol to produce a single AAV vectors carrying both saCas9- and gRNA-expression cassettes was described in the Feng Zhang's group's user manual available online at addgene.org/static/data/plasmids/61/61591/61591-attachment_it03kn5x5O6E.pdf. According to the instructions, CDK1- and PCNA-targeted gRNA sequences were designed, cloned, and annealed into the BsaI-site in an saCas9-AAV vector construct, termed pX601-AAV-CMV::NLS-SaCas9-NLS-3xHA-bGHpA;U6::BsaI-sgRNA. The full nucleotide sequence for the pX601-AAV-CMV::NLS-SaCas9-NLS-3xHA-bGHpA;U6::BsaI-sgRNA plasmid is set forth in FIG. 6.

For CDK1-targeted gRNA generation, two guide sequences were synthesized: CDK1-F: CACCGTCAGACTAGAAAGTGAAGAGGA (SEQ ID NO:11); and CDK1-R: AAACTCCTCTTCACTTTCTAGTCTGAC (SEQ ID NO:12). For PCNA targeted gRNA, PCNA-F: CACCGTTCAGACTATGAAATGAAGTTG (SEQ ID NO:13) and PCNA-R: AAACCAACTTCATTTCATAGTCTGAAC (SEQ ID NO:14) were used. Two guide sequences were annealed and cloned into the Bsal site in the AAV-saCas9 plasmid, pX601-AAV-CMV::NLS-SaCas9-NLS-3xHA-bGHpA;U6::BsaI-sgRNA. Infectious AAV vectors were made using a standard three plasmid transfection method in 293T cells, using pHelper and pRep2Cap2 (Stratagenes), along with one of the saCas9/gRNA-expressing AAV vector plasmids.

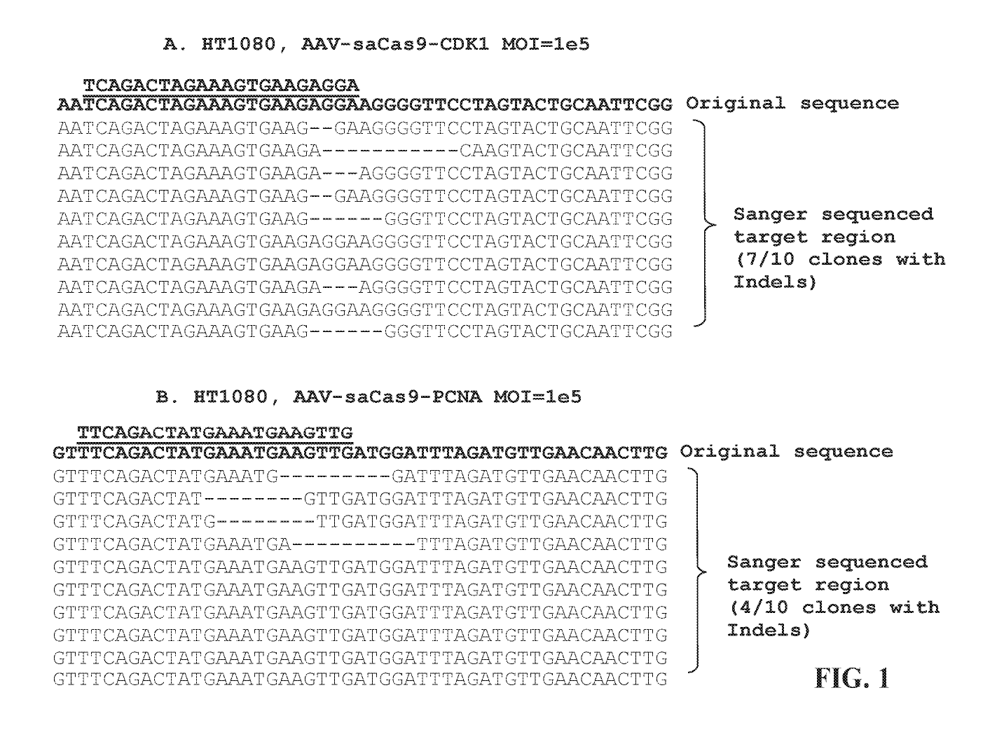

To confirm CDK1- and PCNA-targeted AAV-saCas9-gRNA vectors induced site-directed gene deletions in the gRNA-recognition region, HT1080 cells were infected at a multiplicity of infection (MOI)=1×10⁵ genome copies (gc). Three days after infection, cellular DNA samples were isolated, and the target regions were PCR amplified and cloned. Single clones were sequenced. In the CDK1 vector-treated cells, 70% of PCR clones had target-site-directed Indels (FIG. 1A). In the PCNA vector-treated cells, 40% of PCR clones showed Indels (FIG. 1B). These results demonstrated the feasibility of targeting cell-cycle-associated genes by CRISPR-gRNA vectors.

The following was performed to confirm that viruses designed to express the components of a CRISPR/Cas9 system targeting CDK1 nucleic acid or PCNA1 nucleic acid can reduce cancer cell proliferation. HeLa and HT1080 cells were seeded in 96 well plates at a density of 5000 cells/well. The cells were infected for four consecutive days at MOI of 1×10⁵ with either the AAV-CRISPR/saCas9-CDK1 viruses or the AAV-CRISPR/saCas9-PCNA1. On day 9 from the start, the number of cells was counted, and the cells expanded into 12-well plates. On day 12, the number of cells was counted again. Each condition was performed in triplicate.

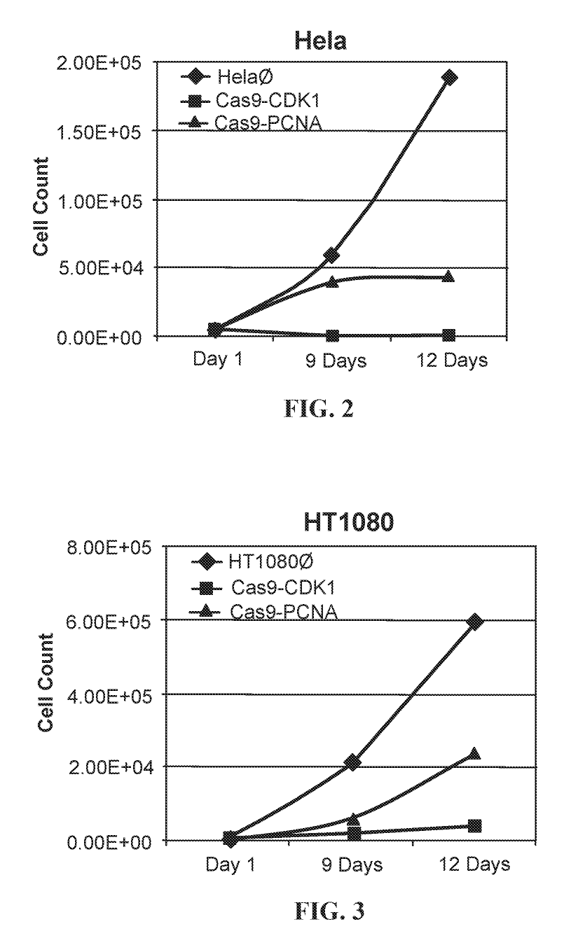

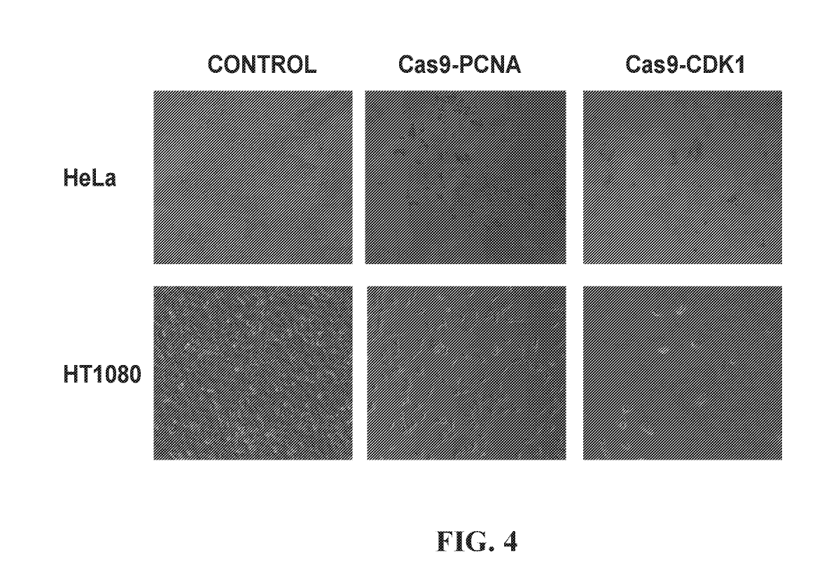

CDK1 targeting with CRISPR/saCas9 blocked cell division of HeLa and HT1080 cells, while PCNA1 targeting delayed cell proliferation (FIGS. 2-4).

These results demonstrate that a CRISPR/Cas9 system targeting CDK1 nucleic acid or PCNA1 nucleic acid can be used to reduce cancer cell proliferation.

### Example 2

### Designing CRISPR/Cas9 Systems that Targets Alu, HERV-K, and HERV-9 Nucleic Acid

AAV vectors were designed to express an saCas9 polypeptide and a targeting guide RNA of a CRISPR/Cas9 system for Alu, HERV-K and HERV-9 sequences, as described in Example 1. Briefly, Alu-, HERV-K- and HERV-9-targeted gRNA sequences were designed, cloned, and annealed into the Bsal-site of pX601-AAV-CMV::NLS-SaCas9-NLS-3xHA-bGHpA;U6::BsaI-sgRNA. gRNA-expressing AAV vectors were produced by a three plasmid transfection method in 293T cells.

Guide sequences for Alu-targeted gRNA generation with the following sequences were synthesized. Alu-F: CACCGCACTTTGGGAGGCCGAGGCGG (SEQ ID NO:15) and Alu-R: AAACCCGCCTCGGCCTCCCAAAGTGC (SEQ ID NO:16).

Two pairs of gRNA sequences for HERV-9 with the following sequences were synthesized. HERV9-F1: CACCGATGTGGGTGGGGCCAGATAA (SEQ ID NO:17) and HERV9-R1: AAACTTATCTGGCCCCACCCACATC (SEQ ID NO:18); as well as HERV9-F2: CACCGAGCCAGCAGTGGCAACCCGC (SEQ ID NO:19) and HERV9-R2: AAACGCGGGTTGCCACTGCTGGCTC (SEQ ID NO:20).

Two pairs of gRNA sequences for HERV-K with the following sequences were synthesized. HERVK-F1: CACCGCGTTTCAGAGAGCACGGGGTT (SEQ ID NO:21) and HERVK-R1: AAACAACCCCGTGCTCTCTGAAAC (SEQ ID NO:22); as well as HERVK-F2: CACCGTCCCTGGGTACTTGAGATTA (SEQ ID NO:23) and HERVK-R2: AAACTAATCTCAAGTACCCAGGGAC (SEQ ID NO:24).

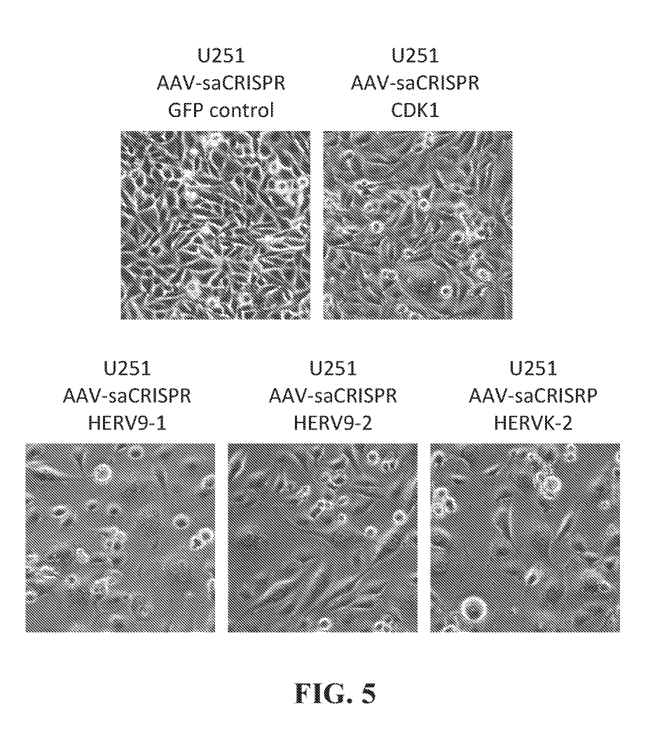

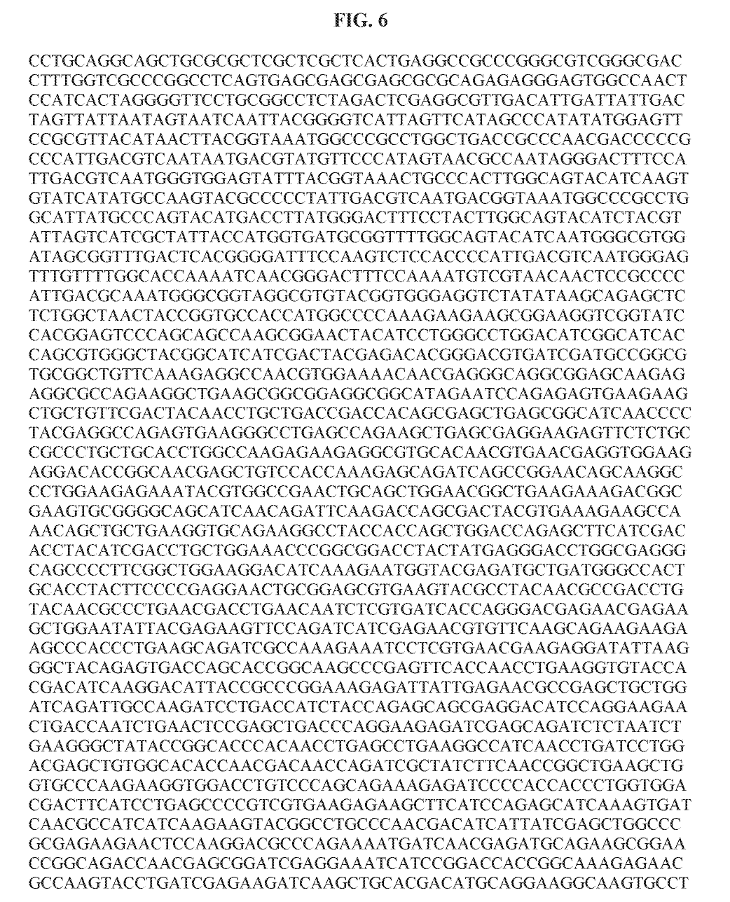

To confirm anti-proliferative effects of HERV-9- and HERV-K-targeted AAV vectors, U251 cells were seeded in 96 well plates at a density of 5000 cells/well. The cells were infected for three consecutive days at MOI of 1×10⁵ with either the AAV-CRISPR/saCas9-HERV-9 viruses or the AAV-CRISPR/saCas9-HERV-K viruses. On day 6, images of infected cells were assessed (FIG. 5), which showed prominent anti-proliferative effects of the vectors. The influence of Alu-, HERV-K-and HERV-9-targeted vectors on cell numbers and cell proliferation rates in U251, HT1080 and HeLa cells, is monitored as described in Example 1.

### Example 3

### Use of CRISPR/Cas9 Systems Targeting CDK1, HERV-9, or HERV-K to Reduce the Number of Cancer Cells within Mammals

Cancer xenograft mouse models were used for in vivo cancer treatment experiments.

AAV vectors were designed to express an saCas9 polypeptide and a targeting guide RNA of a CRISPR/Cas9 system for CDK1 as described in Example 1.

5.00E+06 human fibrosarcoma cells (HT1080) cells in PBS were subcutaneously (S.C) injected into the right flank of SCID beige 6 weeks old mice (N=7). One week later, after establishment of tumors (about 0.2-0.3 cm³), the tumors were injected with a control, no Cas9 AAV vector (AAV2-MCS) at MOI=2.00E+09 VG/g or AAV2-Cas9-CDK1 at MOI=4.00E+08 VG/g, every other day for five times. Tumor volume was measured three times/week.

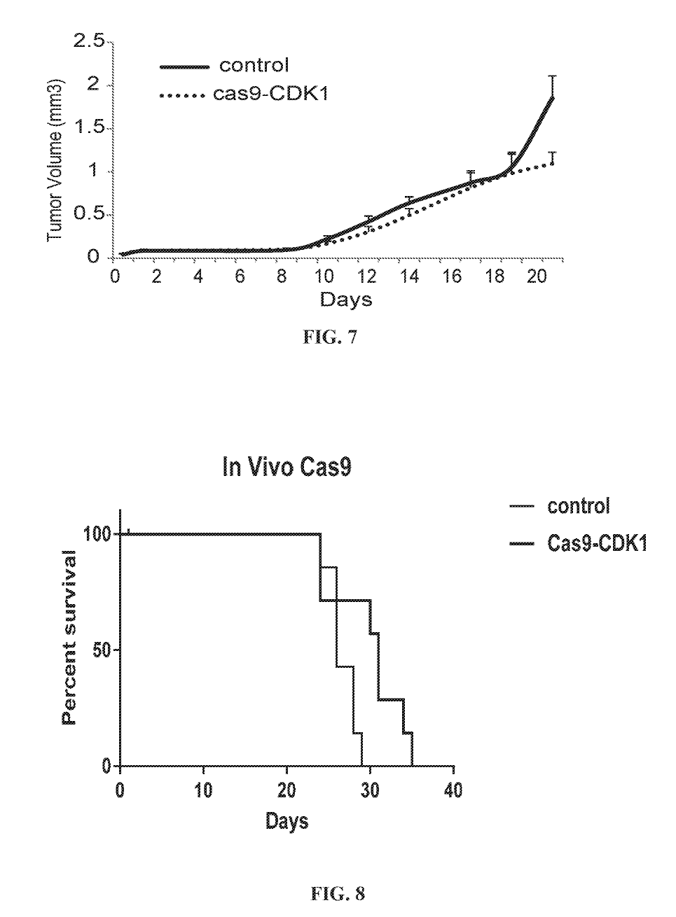

AAV2-Cas9-CDK1 reduced tumor volume sizes (FIG. 7), and extended the survival of treated mice (FIG. 8).

### Example 4

### Use of CRISPR/Cas9 Systems Targeting CDK1, HERV-9, or HERV-K to Reduce the Number of Cancer Cells within Mammals

Cancer xenograft mouse models were used for in vivo cancer treatment experiments.

AAV vectors were designed to express an saCas9 polypeptide and a targeting guide RNA of a CRISPR/Cas9 system for CDK1, HERV-9 and HERV-K sequences, as described in Example 1.

Hela cells in PBS were subcutaneously (SC) injected into the right flank of SCID beige 6 weeks old mice (N=7). One week later, after establishment of tumors (about 0.2-0.3 cm³), the tumors were injected with PBS as a control, AAV2-Cas9-CDK1, AAV2-Cas9-HERV9, or AAV2-Cas9-HERVK at MOI=7.00E+08 VG/g, every other day for eight times. Tumor volume was measured three times/week.

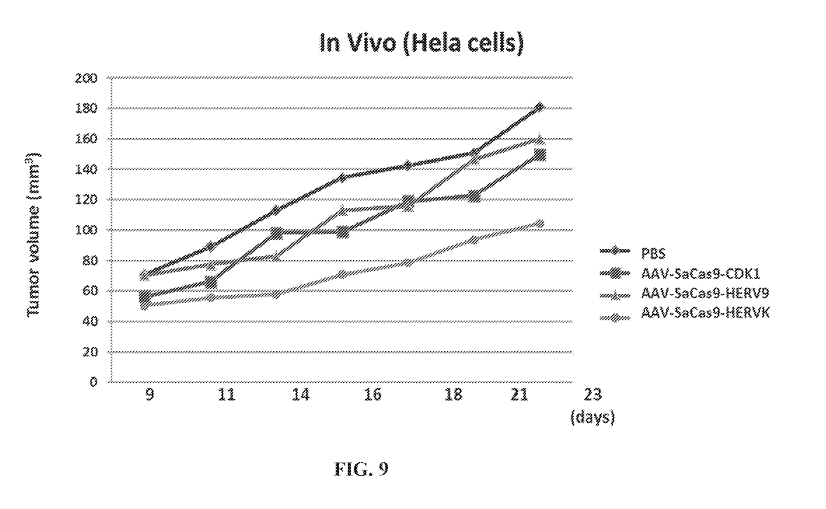

AAV2-Cas9-CDK1, AAV2-Cas9-HERV9, and AAV2-Cas9-HERVK all reduced tumor volume sizes (FIG. 9).

### Example 5

### Use of CRISPR/Cas9 Systems Targeting CDK1 Nucleic Acid or PCNA Nucleic Acid to Reduce the Number of Cancer Cells within Mammals

Cancer xenograft mouse models are used for in vivo cancer treatment experiments. Brain, breast, and pancreatic cancer cell lines are subcutaneously transplanted into immunocompromised mice. After establishment of 1 cm³ tumors, CDK1- and PCNA-targeted AAV vectors are injected every 3 days for 3 weeks, and tumor growth and survival rates are monitored.

### Example 6

### Use of CRISPR/Cas9 Systems Targeting Alu, HERV-K, or HERV-9 to Reduce the Number of Cancer Cells within Mammals

Cancer xenograft mouse models are used for in vivo cancer treatment experiments. Brain, breast, and pancreatic cancer cell lines are subcutaneously transplanted into immunocompromised mice. After establishment of 1 cm³ tumors, Alu-, HERV-K- and HERV-9-targeted AAV vectors are injected every 3 days for 3 weeks, and tumor growth and survival rates are monitored.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nucleic acid construct comprising a nucleic acid encoding a Cas9 polypeptide and a nucleic acid encoding a targeting guide RNA, wherein said targeting guide RNA targets a cell cycle gene or a repetitive nucleic acid sequence.

2. The nucleic acid construct of claim 1, wherein said Cas9 polypeptide is a saCas9 polypeptide.

3. The nucleic acid construct of claim 1, wherein said targeting guide RNA targets said cell cycle gene.

4. The nucleic acid construct of claim 3, wherein said cell cycle gene is CDK1 or PCNA1.

5. The nucleic acid construct of claim 1, wherein said targeting guide RNA targets said repetitive nucleic acid sequence.

6. The nucleic acid construct of claim 5, wherein said repetitive nucleic acid sequence is an Alu nucleic acid sequence, an HERV-K nucleic acid sequence, or an HERV-9 nucleic acid sequence.

7. A viral vector comprising a nucleic acid encoding a Cas9 polypeptide and a nucleic acid encoding a targeting guide RNA, wherein said targeting guide RNA targets a cell cycle gene or a repetitive nucleic acid sequence.

8. The viral vector of claim 7, wherein said Cas9 polypeptide is a saCas9 polypeptide.

9. The viral vector of claim 7, wherein said targeting guide RNA targets said cell cycle gene.

10. The viral vector of claim 9, wherein said cell cycle gene is CDK1 or PCNA1.

11. The viral vector of claim 7, wherein said targeting guide RNA targets said repetitive nucleic acid sequence.

12. The viral vector of claim 11, wherein said repetitive nucleic acid sequence is an Alu nucleic acid sequence, an HERV-K nucleic acid sequence, or an HERV-9 nucleic acid sequence.

13. The viral vector of claim 7, wherein said viral vector is an AAV.

14. A method for reducing the number of cancer cells within a mammal having cancer, wherein said method comprises administering, to said mammal, a nucleic acid construct of claim 1.

15. A method for reducing the number of cancer cells within a mammal having cancer, wherein said method comprises administering, to said mammal, a viral vector of claim 7.


SEQUENCE LISTING (48 sequences)

SEQ ID NO: 1 - DNA, 175 bp, Homo sapiens
gccagatgcggtggctcacgcctgtaatcccagcactttgggaggctgaggcagttggatcacctgaggtcaggaattagcaccactgcactccagcctaggcgacgagagcaaaactctgtctcaaaaaaaaaaaaaaagaaagaaagaaaaaagaaagggctaggagctacaa

SEQ ID NO: 2 - DNA, 26 bp, Homo sapiens
cctgtaatcccagcactttgggaggc

SEQ ID NO: 3 - DNA, 168 bp, Homo sapiens
aaaagtaaaaagaggggccaggcacagtggctcagcctgtaatcccagcactttgggaggctgaggtgggcaggatcacctgagctcgggaagttgaggctaatagtgggctgagattgtgccactgcactccagcctgggtgacagggaaggagaccctgtctcaaa

SEQ ID NO: 4 - DNA, 166 bp, Homo sapiens
ggccaggcatggtgctcatcgcctgtaatcccagcactttgggaggccgagaaagatggatgaagtcaggagttcaagaccagcctgggcaacatggcagaaccccgtctctactaaaaatacaaaaaattagccgggcgtggtggtgggcgcctgtaatcccagc

SEQ ID NO: 5 - DNA, 288 bp, Homo sapiens
ggccgggcgcggtggctcacgcctgtaatcccagcactttgggaggccgaggcgggcggatcacgaggtcaggagatcgagaccatcccggctaaaacggtgaaaccccgtctctactaaaaatacaaaaattagccgggcgtagtggcgggcgcctgtagtcccagctacttgggaggctgaggcaggagaatggcgtgaacccgggaggcggagcttgcagtgagccgagatcccgccactgcactccagcctgggcgacagagcgagactccgtctcaaaaaaaa

SEQ ID NO: 6 - DNA, 45 bp, Homo sapiens
ctgttaatctatgaccttacccccaaccccgtgctctctgaaacg

SEQ ID NO: 7 - DNA, 47 bp, Homo sapiens
ccttaagagtcatcaccactccctaatctcaagtacccagggacaca

SEQ ID NO: 8 - DNA, 40 bp, Homo sapiens
caggatgtgggtggggccagataagagaataaaagcaggc

SEQ ID NO: 9 - DNA, 40 bp, Homo sapiens
tgcccgagccagcagtggcaacccgctcgggtccccttcc

SEQ ID NO: 10 - DNA, 7446 bp, Artificial Sequence
  nucleic acid sequence of pX601-AAV-CMV::NLS-SaCas9-NLS-3xHA-bGHpA;U6::BsaI-sgRNA plasmid
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggagaccacggcaggtctcagttttagtactctggaaacagaatctactaaaacaaggcaaaatgccgtgtttatctcgtcaacttgttggcgagatttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttataagggattttgccgatttcggtctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 27 bp, Artificial Sequence
  forward CDK1 guide sequence
caccgtcagactagaaagtgaagagga

SEQ ID NO: 12 - DNA, 27 bp, Artificial Sequence
  reverse CDK1 guide sequence
aaactcctcttcactttctagtctgac

SEQ ID NO: 13 - DNA, 27 bp, Artificial Sequence
  forward PCNA guide sequence
caccgttcagactatgaaatgaagttg

SEQ ID NO: 14 - DNA, 27 bp, Artificial Sequence
  reverse PCNA guide sequence
aaaccaacttcatttcatagtctgaac

SEQ ID NO: 15 - DNA, 26 bp, Artificial Sequence
  forward Alu guide sequence
caccgcactttgggaggccgaggcgg

SEQ ID NO: 16 - DNA, 26 bp, Artificial Sequence
  reverse Alu guide sequence
aaacccgcctcggcctcccaaagtgc

SEQ ID NO: 17 - DNA, 25 bp, Artificial Sequence
  forward HERV9 guide sequence
caccgatgtgggtggggccagataa

SEQ ID NO: 18 - DNA, 25 bp, Artificial Sequence
  reverse HERV9 guide sequence
aaacttatctggccccacccacatc

SEQ ID NO: 19 - DNA, 25 bp, Artificial Sequence
  forward HERV9 guide sequence
caccgagccagcagtggcaacccgc

SEQ ID NO: 20 - DNA, 25 bp, Artificial Sequence
  reverse HERV9 guide sequence
aaacgcgggttgccactgctggctc

SEQ ID NO: 21 - DNA, 26 bp, Artificial Sequence
  forward HERVK guide sequence
caccgcgtttcagagagcacggggtt

SEQ ID NO: 22 - DNA, 24 bp, Artificial Sequence
  reverse HERVK guide sequence
aaacaaccccgtgctctctgaaac

SEQ ID NO: 23 - DNA, 25 bp, Artificial Sequence
  forward HERVK guide sequence
caccgtccctgggtacttgagatta

SEQ ID NO: 24 - DNA, 25 bp, Artificial Sequence
  reverse HERVK guide sequence
aaactaatctcaagtacccagggac

SEQ ID NO: 25 - DNA, 22 bp, Homo sapiens
tcagactagaaagtgaagagga

SEQ ID NO: 26 - DNA, 49 bp, Homo sapiens
aatcagactagaaagtgaagaggaaggggttcctagtactgcaattcgg

SEQ ID NO: 27 - DNA, 47 bp, Artificial Sequence
  CDK1 sequence with site-directed gene deletion
aatcagactagaaagtgaaggaaggggttcctagtactgcaattcgg

SEQ ID NO: 28 - DNA, 38 bp, Artificial Sequence
  CDK1 sequence with site-directed gene deletion
aatcagactagaaagtgaagacaagtactgcaattcgg

SEQ ID NO: 29 - DNA, 46 bp, Artificial Sequence
  CDK1 sequence with site-directed gene deletion
aatcagactagaaagtgaagaaggggttcctagtactgcaattcgg

SEQ ID NO: 30 - DNA, 47 bp, Artificial Sequence; identical to SEQ ID NO: 27
  CDK1 sequence with site-directed gene deletion

SEQ ID NO: 31 - DNA, 43 bp, Artificial Sequence
  CDK1 sequence with site-directed gene deletion
aatcagactagaaagtgaaggggttcctagtactgcaattcgg

SEQ ID NO: 32 - DNA, 49 bp, Artificial Sequence; identical to SEQ ID NO: 26
  CDK1 sequence with site-directed gene deletion

SEQ ID NO: 33 - DNA, 49 bp, Artificial Sequence; identical to SEQ ID NO: 26
  CDK1 sequence with site-directed gene deletion

SEQ ID NO: 34 - DNA, 46 bp, Artificial Sequence; identical to SEQ ID NO: 29
  CDK1 sequence with site-directed gene deletion

SEQ ID NO: 35 - DNA, 49 bp, Artificial Sequence; identical to SEQ ID NO: 26
  CDK1 sequence with site-directed gene deletion

SEQ ID NO: 36 - DNA, 43 bp, Artificial Sequence; identical to SEQ ID NO: 31
  CDK1 sequence with site-directed gene deletion

SEQ ID NO: 37 - DNA, 22 bp, Homo sapiens
ttcagactatgaaatgaagttg

SEQ ID NO: 38 - DNA, 49 bp, Homo sapiens
gtttcagactatgaaatgaagttgatggatttagatgttgaacaacttg

SEQ ID NO: 39 - DNA, 40 bp, Artificial Sequence
  PCNA sequence with site-directed gene deletion
gtttcagactatgaaatggatttagatgttgaacaacttg

SEQ ID NO: 40 - DNA, 41 bp, Artificial Sequence
  PCNA sequence with site-directed gene deletion
gtttcagactatgttgatggatttagatgttgaacaacttg

SEQ ID NO: 41 - DNA, 41 bp, Artificial Sequence; identical to SEQ ID NO: 40
  PCNA sequence with site-directed gene deletion

SEQ ID NO: 42 - DNA, 39 bp, Artificial Sequence
  PCNA sequence with site-directed gene deletion
gtttcagactatgaaatgatttagatgttgaacaacttg

SEQ ID NO: 43 - DNA, 49 bp, Artificial Sequence; identical to SEQ ID NO: 38
  PCNA sequence with site-directed gene deletion

SEQ ID NO: 44 - DNA, 49 bp, Artificial Sequence; identical to SEQ ID NO: 38
  PCNA sequence with site-directed gene deletion

SEQ ID NO: 45 - DNA, 49 bp, Artificial Sequence; identical to SEQ ID NO: 38
  PCNA sequence with site-directed gene deletion

SEQ ID NO: 46 - DNA, 49 bp, Artificial Sequence; identical to SEQ ID NO: 38
  PCNA sequence with site-directed gene deletion

SEQ ID NO: 47 - DNA, 49 bp, Artificial Sequence; identical to SEQ ID NO: 38
  PCNA sequence with site-directed gene deletion

SEQ ID NO: 48 - DNA, 49 bp, Artificial Sequence; identical to SEQ ID NO: 38
  PCNA sequence with site-directed gene deletion